(19) United States
(12) Patent Application Publication
(10) Pub. No.: US 2006/0034953 A1
(43) Pub. Date: Feb. 16, 2006

(54) Ayurvedic nutritional preparation

(76) Inventor: Anthony Devasia Joseph, Pune (IN)

Correspondence Address: MERCHANT & GOULD PC, P.O. BOX 2903, MINNEAPOLIS, MN, 55402-0903, US

(21) Appl. No.: 10/519,879

(22) PCT Filed: Jun. 23, 2003
(86) PCT No.: PCT/IN03/00331

§ 371 Date: Dec. 30, 2004

(30) Foreign Application Priority Data
Jun. 24, 2002 (IN) 522/MUM/2002
Jul. 19, 2002 (IN) 655/MUM/2002

(51) Int. Cl. A61K 36/9068; A61K 36/8962

(52) U.S. Cl. 424/754; 424/756; 424/769

(57) ABSTRACT

The invention relates to an ayurvedic nutricinal preparation used as a prophylactic for cardiac ailments and for increasing cardiac muscle tonicity. The principal ingredient used are nuts from the plants belonging to the anacardiacieae family, Allium Sativum Linn and Zingiber Officinale Rosc. The toxins and irritants from the nuts are removed or deactivated, before the oil is extracted from the nuts. The formulation is prepared with the filtered nut oil extract, and an extract of Allium Sativum Linn and Zingiber Officinale Rosc.

### FIELD OF INVENTION

The invention relates to an ayurvedic nutricinal preparation, essentially from nuts of the anacardiaciae family, Allium Sativum Linn and Zingiber Officinale Rosc for use as a prophylatic cardiac tonic, for general revitalization of the human body and for increasing cardiac muscle tonicity.

### BACKGROUND OF THE INVENTION

Several ayurvedic preparations are in use for the treatment of heart ailments and as a prophylatic preparation for cardiac care. Most commonly used and reported are extracts or preparations from the plant Tenninalia aijuna. Patent Application No: 1184/MUM/2001 describes a process for an ayurvedic preparation from Term inalia aijuna bark, Acorus calmus stem, Magnifera indica bark, Moringe oelifera bark, Murraya koengil leaf, Pipeer longum fruit, Boerhaavia diffusa root. The presently known ayurvedic preparations are very selectively effective.

The present invention describes for the first time a formulation for a complete ayurvedic preparation, made essentially from nuts of plants belonging to the genus anacardiaciae, for revitalising the human body. The preparation enhances the tonicity of the heart muscles, increases the production of blood by the marrow, reduces occlusion of arteries, improves myocardial blood flow and prevents cardiac ailments.

Nuts from the anacardiaciae family contain toxins and irritants, and cause contact dermatitis, because of which rash and irritation occurs. Hence its nutricinal value has been explored only in a limited way for cancer, gastric infections and fungal infections but not in respect of cardiovascular treatment. The invention also describes for the first time the use of nuts of the anacardiaciae family from which the toxic irritants are removed, and is combined with the extract of Allium Sativum Linn and Zingiber Officinale Rosc for treatment of cardiac ailments.

### SUMMARY

In its main aspect the invention consists of an ayurvedic nutricinal preparation for use as a prophylactic for cardiac ailments and for increasing cardiac muscle tonicity. The principal ingredient used is a nut oil extract prepared from clean and dried nuts from plants of the genus anacardiaciae, and an extract of Allium Sativum Linn and Zingiber Officinale Rose in vegetable oil. To obtain a nut oil extract, the nuts are cleaned and dried and the toxins and irritants contained in the nuts, are removed or deactivated by repeated cooking and drying of the nuts in milk to obtain pure detoxified nuts. The oil in the nuts which is the essential ingredient for this invention, is extracted by boiling the detoxified nuts in a medium of clarified butter, preferably obtained from cow's milk, until all the moisture is removed. In another aspect of this invention, 5 proportions by weight of ghee is used for every 2 proportions by weight of nuts. The medium is maintained at a temperature of 50° to 80°, for an extended period of time until the medium is absolutely moisture free. An extract of Allium Sativum Linn and Zingiber Officinale Rosc is obtained by cooking the two in a moisture free vegetable oil. The nut oil extract and the Allium Sativum Linn and Zingiber Officinale Rosc extract are filtered separately and mixed together.

In another aspect of this invention, the nuts used are either of the species Semecarpous Anacardium Linn or Anacardium Occidenatale Linn.

In a further aspect of this invention Zingiber Officinale Rose and Allium Sativum Linn are used in a proportion of 1:2 by weight in vegetable oil of 3 proportions by weight, and mixed with filtered nut oil extract obtained from 2 proportions by weight of nuts.

In yet another aspect of this invention the vegetable oil used is purified oil from Ricinus Conmmunis Linn.

In a final aspect of this invention, the ayurvedic nutricinal preparation as described above is made available in soft gelatin capsules.

### DESCRIPTION

The following description of the formulation is the best known method of working this invention.

In its main embodiment the invention consists of a nutricinal preparation for use as a prophylactic for cardiac elements and for increasing cardiac muscle tonicity.

It comprises essentially of
i) a nut oil extract in clarified butter. The nut oil extract is obtained from clean, dried and processed nuts from the plants of the genus anacardiaciae.
ii) An extract of Allium Sativum Linn and Zingiber Officinale Rose, which is prepared in moisture free vegetable oil

The nuts of the anacardiaciae family are cleaned and dried. Subsequently they are cooked in milk and dried. The process is repeated several times to obtain pure detoxified nuts from which the irritants and toxins have been removed or deactivated. Nuts preferably used are from the plants of Semecarpous Anacardium Linn or Anacardium Occidenatale Linn. The detoxified nuts are boiled in a medium of clarified butter (ghee), until all the moisture is removed and the spluttering of the medium ebbs. Clarified butter made from cow's milk is preferred. For every 2 proportion by weight of nuts, 5 proportion by weight of ghee is used. The medium is maintained at a temperature of 50° to 80° for an extended period of time to ensure removal of any remainder moisture. By the aforesaid process the essential oil from the nut is extracted in to the medium By a separate process, Zingiber Officinale Rosc and Allium Sativum Linn in a proportion of 1:2 by weight is cooked in purified moisture free vegetable oil, preferably oil from Ricinus Communis Linn, to obtain a Zingiber Officinale Rose—Allium Sativum Linn extract. The Zingiber Officinale Rosc—Allium Sativum Linn extract and the nut oil extract are separately filtered to remove all solid particles and mixed together.

In the preferred embodiment of this invention, 1:2 proportion by weight of Zingiber Officinale Rose and Allium Sativum Linn are cooked in 3 proportions by weight of vegetable oil. The filtered extract of Zingiber Officinale Rose and Allium Sativum Linn so obtained is mixed with filtered nut oil extract obtained from 2 proportions by weight of nuts.

The viscous liquid is ready for use in doses proportional to age and body weight of the consumer. The viscous liquid may also be encapsulated in soft gelatin capsules for dispensing.

The following are examples of some in vivo clinical trials conducted, on the basis of oral administration of said ayurvedic nutricinal preparation:

Comparative Study of I.H.D (Ischemic Heart Disease) Patient Before and
After Administration of CF1 Based on Symptoms and 2D Echo/Color Doppler

| | | | Report of | Period of | | Report of post | |
|---|---|---|---|---|---|---|---|
| | | Symptoms | pretreatment | treatment | Symptoms | treatment | |
| No | diagnosis | pretreatment | 2D Echo | With CF1 | Post treatment | 2D Echo | Remarks |
| 001 | I.H.Dⁱ. | Breathlessness on | Severe global | 1-0 × 90 | NIL | Global | Overall |
| | | mild effort | hypokinesia | | Can walk | hypokinesia | good |
| | | Severe chest pain | Severe LVⁱⁱⁱ | | comfortably 2-3 | reduced | improvement |
| | | on walking | dysfunction | | kms. | Moderate LVⁱⁱⁱ | |
| | | Giddiness | Severe PAH^{viii} | | | dysfunction | |
| | | | LVEF^{iv} 15-20% | | | Mild PAH^{viii} | |
| | | | | | | LVEF^{iv} 34% | |
| 003 | I.H.D. | Angina on mild | Distal IVS^{x}, | 1-0 × 60 | Angina | No regional wall | Overall |
| | | effort | apex and | | reduced | motion | good |
| | | breathlessness on | anterior wall | | Breathlessnsss | abnormality at rest | improvement |
| | | effort | hypokinetic | | reduced to | Normal atrio- | Maintained |
| | | unable to climb | Trivial MR^{v}, TR^{vi} | | marked extent | ventricular and | good health |
| | | stairs | and PR^{vii} | | | semilunar valves | |
| | | | LVEF^{iv} 50% | | | LVEF^{iv} 60% | |
| | | | | | | Normal LVⁱⁱⁱ | |
| | | | | | | systolic function | |
| 006 | I.H.Dⁱ. | general debility | Distal IVS^{x} & | 2-0 × 105 | Breathlessness | LVEF −35% | Overall |
| | | Chest pain and | LVⁱⁱⁱ is akinetic | | reduced | Distal IVS^{x} & LVⁱⁱⁱ | good |
| | | breathlessness on | LVEF^{iv} 30% | | chest pain | hypokinetic | improvement |
| | | effort | Evidence of | | occasional | No evidence of | Maintained |
| | | | organised clot | | improved | clot in LA^{ix} or LVⁱⁱⁱ | good health |
| | | | at LVⁱⁱⁱ Apex | | appetite | Normal LVⁱⁱⁱ | |
| | | | LVⁱⁱⁱ, diastolic | | general feeling | diastolic function | |
| | | | dysfunction | | of well being. | | |
| 011 | I.H.D. | Chest discomfort | Ischemic heart | 2-0 × 120 | Chest | Normal sized LV | overall good |
| | | Pain in left arm | disease | | discomfort | Normal systolic & | recovery |
| | | Weakness | Hypokinetic | | reduced | diastolic function | |
| | | | inferior and | | pain in the left | LVEF^{iv} 78% | |
| | | | apical segments | | arm | No mitral valve | |
| | | | LVEF^{iv} 45% | | disappeared | prolapse | |
| | | | | | feeling of well | No segmental | |
| | | | | | being | wall motion | |
| | | | | | | anomaly | |
| 012 | I.H.D. | Breathlessness | Ischemic heart | 2-0 × 90 | No | Normal LV | Overall |
| | | Chest pain on effort | disease-LVEF^{iv} 39% | | breathlessness | function | good |
| | | Heart bum | | | No chest pain | LVEF^{iv} 55% | recovery |
| | | frequently | | | No heart burn - | | |
| | | | | | working 8-10 | | |
| | | | | | hrs a day | | |

ⁱI.H.D—Ischemic Heart Disease
ⁱⁱLV—Left Ventricle
ⁱⁱⁱLVEF—Left Ventricle Hypertrophy
^{iv}M.R.—Mitral Regurgitation
^{v}T.R.—Tricuspid Regurgitation
^{vi}P.R.—Pulmonary Regurgitation
^{vii}P.A.H.—Pulmonary Artery Hypertrophy
^{viii}L.A.—Left Auricle
^{ix}I.V.S.—Inter Ventricular Septum

I claim:

1. An ayurvedic nutricinal preparation for use as a prophylactic for cardiac ailments and for increasing cardiac muscle tonicity comprising essentially of
a. a nut oil extract, with minimal or no toxins and irritants, obtained from cleaned, dried and processed nuts from plants of the genus anacardiaciae;
b. an extract of Allium Sativum Linn'Zingiber Officinale Rosc Linn

2. A medicinal preparation as described in claim 1 where in the nuts are either of the species Semecarpous Anacardium Linn or Anacardium Occidenatale Linn.

3. A medicinal preparation as described in claim 2, where in the nut extract is prepared in clarified butter.

4. A medicinal preparation as described in claim 3, where the nut oil extract in clarified butter is obtained by using 5 proportion by weight of clarified butter for every 2 proportion by weight of nuts.

5. A medicinal preparation as described in claim 4, where in the extract of Allium Sativum Linn and Zingiber Officinale Rosc is prepared by cooking Allium Sativum Linn and Zingiber Officinale Rosc in moisture free vegetable oil

6. A medicinal preparation as described in claim 5, where in the Zingiber Officinale Rosc and Allium Sativum Linn are used in a proportion of 1:2 by weight in vegetable oil of 3 proportions by weight, and mixed with filtered nut oil extract obtained from 2 proportions by weight of nuts.

7. A medicinal preparation as described in claim 6 which is in soft gelatin capsule form.

8. A medicinal preparation as described in claim 2, prepared by a process comprising of steps including:
a. Partial or complete removal of toxins and irritants from cleaned and dried nuts from plants of the genus, anacardiaciae, by repeated cooking and drying of the nuts in milk to obtain pure detoxified nuts;
b. Boiling the detoxified nuts in a medium of clarified butter preferably from cow's milk and maintaining the medium at a temperature of 50° to 80°, for an extended period of time until the medium is absolutely moisture free to obtain a nut oil extract from the nuts;
c. Cooking Allium Sativum Linn and Zingiber Officinale Rosc in moisture free vegetable oil to obtain an Allium Sativum Linn and Zingiber Officinale Rosc extract;
d. Separately filtering the nut oil extract and Allium Sativum Linn-Zingiber Officinale Rosc extract and mixing the two.

9. A process as described in claim 8, where in the vegetable oil used is purified oil from Ricinus Communis Linn.

10. A process as described in claim 9, where in the nut oil extract in clarified butter is obtained by using 5 proportion by weight of clarified butter for every 2 proportion by weight of nuts.

11. A process as described in claim 10, where in the Zingiber Officinale Rosc and Allium Sativum Linn are used in a proportion of 1:2 by weight in vegetable oil of 3 proportions by weight, and mixed with filtered nut oil extract obtained from 2 proportions by weight of nuts.
